(19) United States
(12) Patent Application Publication
(10) Pub. No.: US 2020/0292541 A1
(43) Pub. Date: Sep. 17, 2020

(54) WHOLE BLOOD STAINING PREPARATION CARTRIDGE AND SYSTEM

(71) Applicant: EI SPECTRA, LLC, Ketchum, ID (US)

(72) Inventor: Harold E. Ayliffe, Ketchum, ID (US)

(21) Appl. No.: 16/084,036

(22) PCT Filed: Mar. 15, 2017
(86) PCT No.: PCT/US17/22602

§ 371 Date: Sep. 11, 2018

Related U.S. Application Data
(60) Provisional application No. 62/308,351, filed on Mar. 15, 2016.

(51) Int. Cl. G01N 33/555; B01L 3/00; G01N 15/12; G01N 15/14

(52) U.S. Cl. CPC G01N 33/555; B01L 3/502738; G01N 2015/1006; G01N 15/14; G01N 15/12

(57) ABSTRACT

A device for facilitating volumetric blood-cell counting includes a blood reservoir configured to receive a blood sample, a first rotatable valve including a first duct having first and second ends, and a first container containing a first reagent. The first end of the first duct is configured to be selectively in fluid communication with the blood reservoir and first container. The device further includes a mixing chamber. The second end of the first duct is configured to be selectively in fluid communication with the mixing chamber.

### PRIORITY CLAIM

This patent application claims priority to U.S. Prov. Pat. Appl. No. 62/308,351 filed Mar. 15, 2016, the contents of which are hereby incorporated by reference as if fully set forth herein.

### BACKGROUND

In clinical environments, there are often situations in which a patient requires specific white blood cell (“WBC”) counts to be performed as soon as possible, as the results directly impact downstream decisions. For example, a patient may be undergoing a 4- to 6-hour aphaeresis treatment to extract to hematopoietic progenitor cell antigen CD34 (“CD34”) white blood cells for subsequent re-injection. Just prior to starting aphaeresis, two hours into it, and at the end of treatment it is desirable to test the volumetric counts of the CD34 WBCs. There is currently no practical means for performing a rapid CD34 white blood cell count at or near the patient.

### BRIEF DESCRIPTION OF THE DRAWING

Preferred and alternative embodiments of the present invention are described in detail below with reference to the following drawing figures.

FIG. 1 is a front view of a cartridge according to an embodiment of the invention;

FIG. 2 is a side cross-sectional view of the cartridge of FIG. 1;

FIG. 3 is a front cross-sectional view of the cartridge of FIG. 1:

FIG. 4 is FIG. 2 is a rear view of the cartridge of FIG. 1:

FIGS. 5-8 illustrate an embodiment of a system for use with the cartridge of FIG. 1;

FIGS. 9-10 illustrate a cartridge according to an alternative embodiment of the invention.

### DETAILED DESCRIPTION

This patent application is intended to describe one or more embodiments of the present invention. It is to be understood that the use of absolute terms, such as “must,” “will,” and the like, as well as specific quantities, is to be construed as being applicable to one or more of such embodiments, but not necessarily to all such embodiments. As such, embodiments of the invention may omit, or include a modification of, one or more features or functionalities described in the context of such absolute terms.

Embodiments of the invention may be described in the general context of computer-executable instructions, such as program modules, being executed by a processing device having specialized functionality and/or by computer-readable media on which such instructions or modules can be stored. Generally, program modules include routines, programs, objects, components, data structures, etc. that perform particular tasks or implement particular abstract data types. The invention may also be practiced in distributed computing environments where tasks are performed by remote processing devices that are linked through a communications network. In a distributed computing environment, program modules may be located in both local and remote computer storage media including memory storage devices.

Embodiments of the invention may include or be implemented in a variety of computer readable media. Computer readable media can be any available media that can be accessed by a computer and includes both volatile and nonvolatile media, removable and non-removable media. By way of example, and not limitation, computer readable media may comprise computer storage media and communication media. Computer storage media include volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

According to one or more embodiments, the combination of software or computer-executable instructions with a computer-readable medium results in the creation of a machine or apparatus. Similarly, the execution of software or computer-executable instructions by a processing device results in the creation of a machine or apparatus, which may be distinguishable from the processing device, itself, according to an embodiment.

Correspondingly, it is to be understood that a computer-readable medium is transformed by storing software or computer-executable instructions thereon. Likewise, a processing device is transformed in the course of executing software or computer-executable instructions. Additionally, it is to be understood that a first set of data input to a processing device during, or otherwise in association with, the execution of software or computer-executable instructions by the processing device is transformed into a second set of data as a consequence of such execution. This second data set may subsequently be stored, displayed, or otherwise communicated. Such transformation, alluded to in each of the above examples, may be a consequence of, or otherwise involve, the physical alteration of portions of a computer-readable medium. Such transformation, alluded to in each of the above examples, may also be a consequence of, or otherwise involve, the physical alteration of, for example, the states of registers and/or counters associated with a processing device during execution of software or computer-executable instructions by the processing device.

As used herein, a process that is performed “automatically” may mean that the process is performed as a result of machine-executed instructions and does not, other than the establishment of user preferences, require manual effort.

An embodiment of the invention includes a whole-blood cell prep cartridge. This cartridge and corresponding system provides an automated means to prepare whole blood for subsequent white blood cell analysis, specifically using flow cytometry. The disclosed blood cartridge also can safely store fluorescent and biological reagents in the cartridge. In order to do this, an embodiment of the invention includes metabolized Mylar fluid pouches. The pouches can hold the required volumes, are liquid- and air-tight, and are opaque (which aids to protect the fluorescent dyes). The disclosed whole-blood cell prep cartridge allows a nurse or lab technician to simply and repeatably prepare a patient's whole blood to allow for subsequent volumetric counting using a flow cytometer.

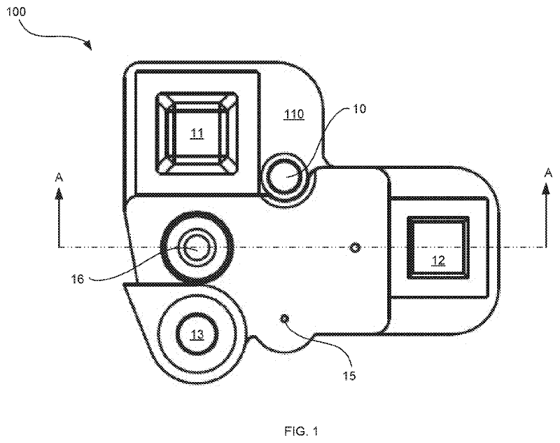

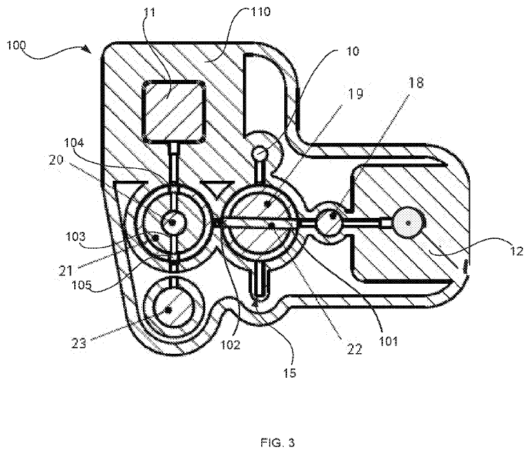

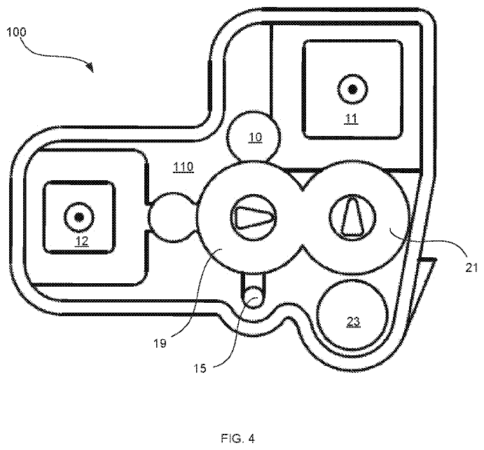

As illustrated in FIGS. 1-4, an embodiment of the invention includes an automated, mesoscale fluidic prep cartridge 100 that includes all of the components necessary to fluorescently label the desired white blood cells for subsequent volumetric counting using a flow cytometer. Specifically, the cartridge 100 can accurately select a volume of blood from a surplus of blood. This ensures accurate downstream volumetric WBC counts. To accomplish this, an embodiment of the invention includes a rotating valve mechanism to select a volume of blood and make it available for subsequent mixing with a fluorescent dye mixture. Specifically, cartridge 100 includes a fluid (e.g., blood) reservoir 10, which in a preferred embodiment has a volume of 100 μL, configured to receive a blood sample. A rotating first valve 19 contains a duct 22, which may be a tube, having a first end 101 and a second end 102. The first end 101 of the duct 22 can be positioned in fluid communication with the blood reservoir 10 by rotating first valve 19. Cartridge 100 further includes an integrated hydrophobic vent/port 15. As is discussed in greater detail herein, the hydrophobic vent technology, which may employ hydrophobic membranes, is used in multiple locations within the whole blood prep cartridge 100 to ensure that trapped air does not cause downstream volumetric count inaccuracies. By applying vacuum to port 15, whole blood can be pulled (or, alternatively, driven via capillary forces) into the cartridge 100 and into duct 22 from reservoir 10 up to port 15 where such blood flow is stopped. The valve 19 may then be rotated 45 degrees from the vertical position (i.e., position in fluid communication with reservoir 10), the duct 22 having thereby collected a known volume of blood. This feature enables a user to obtain accurate counts by starting with a precise, and known, volume of whole blood (in an embodiment, 50 μL).

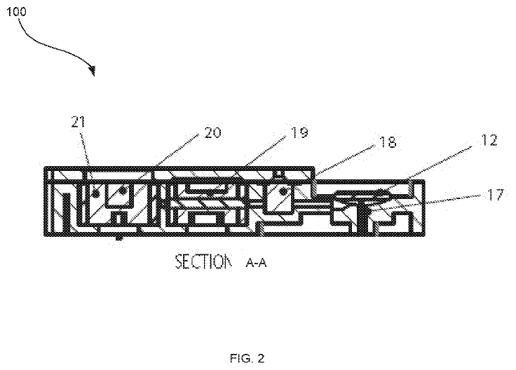

Cartridge 100 further includes a fluorescent liquid dye pouch 12 and an RBC lyse pouch 11. In an embodiment, with the valve 19 still at 45 degrees from vertical, the fluorescent dye pouch 12 can be compressed by a piercing mechanism 17 (FIG. 2). The fluid containing the fluorescent dye is thereby released and flows into a de-gassing chamber port 18 that contains a hydrophobic vent. This intermediate chamber 18 allows as much air as possible to be removed to help minimize counting volumetric counting errors.

A rotating second valve 21 includes a duct 103, which may be a tube, having a first end 104 and a second end 105. The first end 104 of the duct 103 can be positioned in fluid communication with the second end 102 of duct 22 by rotating first valve 19. In an embodiment, the valve 19 may be rotated to a horizontal position and a vacuum can be applied to hydrophobic port 16 to draw the blood/dye mixture via duct 103 to a, preferably, 100 μL collection well 20 that is incorporated into the valve 21. The blood/dye mixture may be allowed to incubate in well 20 for a predetermined amount of time to allow the dye to specifically bind to the WBCs of interest.

Once incubation of the blood/dye mixture is complete, pouch 11 filled with, in an embodiment, 900 μL of red blood cell lyse reagent is pierced, and the valve 21 is rotated such that first end 104 is in fluid communication with pouch 11 and second end 105 is in fluid communication with a mixing chamber 23. By applying a vacuum to a hydrophobic port 13, the combination of blood/dye mixture and lyse reagent are drawn into chamber 23 where the combination is stirred with, in an embodiment, a miniature magnetic stir bar and an external electric motor to eliminate as many red blood cells as possible. After mixing for a predetermined amount of time, the mixed sample can be extracted from cartridge 100 for volumetric flow cytometry analysis. In an embodiment, a user can insert the entire cartridge 100 into a custom flow cytometer that will automatically extract the prepared blood directly from the cartridge.

Cartridge 100 further comprises a housing 110 within which the blood reservoir 10, rotatable valves 19, 21, pouches 11, 12, ports 13, 15, 16, 18 and mixing chamber 23 are disposed.

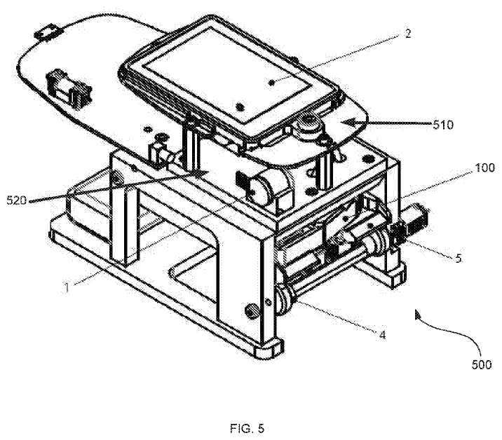

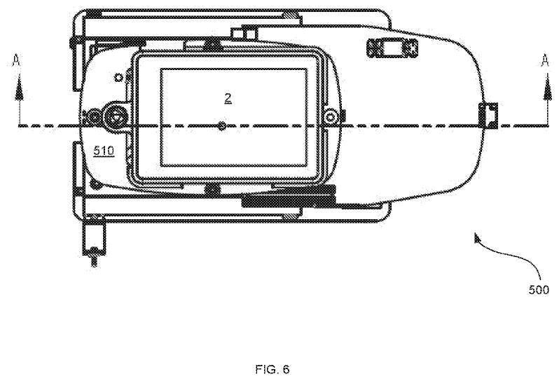

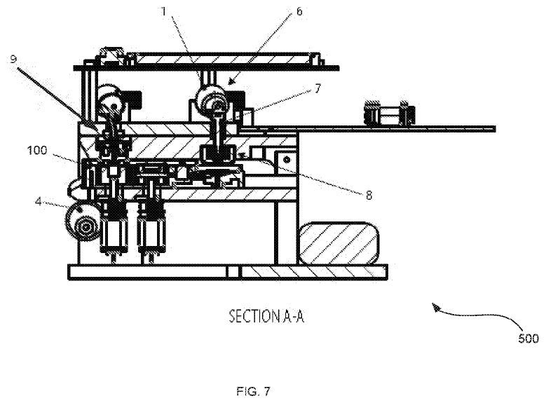

Referring now to FIGS. 5-8, an embodiment of a system 500 for use with the cartridge 100 is completely self-contained and has an integrated, fully programmable digital circuit board 510 with color touch display 2 and an analog circuit board 520. It may further have a battery and a USB cable for charging and power.

An embodiment has five electric gear motors with encoders for position feedback. Two of the motors (one of which is illustrated as element 1) are used to pierce the liquid pouches 11, 12 in the cartridge 100 via mechanical cam 6 and a spring-loaded follower type mechanism 7. More specifically, a spring acts to bias follower 7 and associated piston/cylinder 8 upwards against cam 6 that is connected to indexing gear motor 1. When the gear motors 1 are activated, the cams 6 rotate causing the follower 7 and piston 8 to compress the liquid-containing pouches 11, 12. Two other motors are used to rotate the fluid valves 19, 21. One motor 4 is used (optionally) to provide an automated cartridge tray opening/closing motion. The system 500 may also have a sixth electric gear motor with no encoder for turning the magnetic stir bar inside the mixing chamber 23.

The pneumatic structure 9 of the system 500 has three spring loaded “pistons” with O-rings that apply a controlled force on the cartridge 100 once the tray 5 is loaded. Each piston is connected to the pneumatic system via a flexible tube. An embodiment uses vacuum only, but positive pressure can also be used to move fluids within the cartridge 100. The pneumatic structure 9 has a pump, a pressure transducer, a vacuum reservoir (optional), and four solenoid vales. Three solenoid values are used to direct the vacuum source to each of the three pistons. The fourth solenoid valve may be used to vent the system.

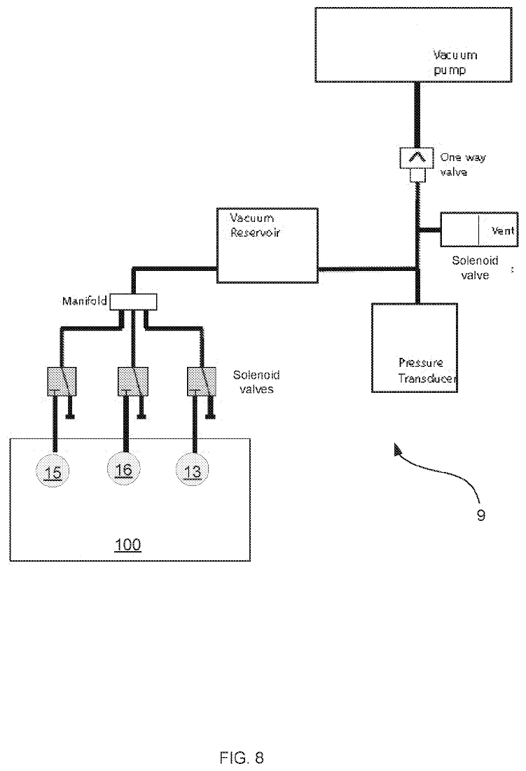

FIG. 8 depicts a schematic for the pneumatic system 9 that can be used in the system that interfaces with the cartridge 100. System 9 can generate the vacuum pressure necessary to move the fluids around inside the cartridge 100.

Vacuum pressure is directed to the cartridge 100 one port 13, 15, 16 at a time via activation of one of the three solenoid valves shown. The remainder of the system may comprise one or more combinations of a vacuum pump, a reservoir, a pressure transducer to measure the generated pressure, and a fourth solenoid to vent the cartridge 100 when needed.

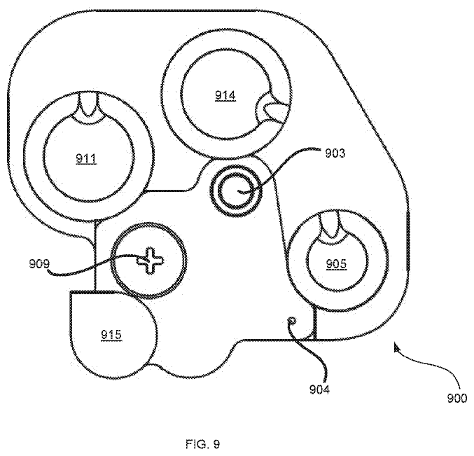

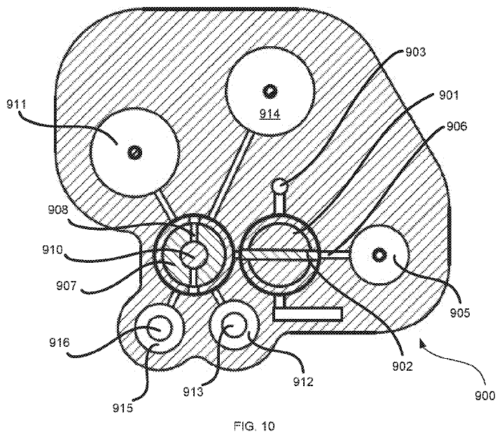

An alternative embodiment cartridge 900, illustrated in FIGS. 9-10, includes a magnetic microbead-based cartridge. As discussed below, cartridge 900 employs many of the same concepts/structures as that of cartridge 100, but incorporates magnetic micro particles to isolate the cells using an externally applied permanent magnet and multiple wash steps.

In an embodiment, a first valve 901 is placed in a vertical position such that a duct 902 is in fluid communication with a blood reservoir 903. Vacuum is applied to hydrophobic port 904 until blood from reservoir 903 reaches port 904. Valve 901 may then be rotated 45 degrees. Reagent pouch 905 containing fluorescent dye and magnetic particles is pierced and compressed, and this reagent flows into the reagent/de-gassing chamber port 906 through which air may be released. Mechanical pressure on pouch 905 is maintained as valve 901 is rotated to a horizontal position such that duct 902 is in fluid communication with port 906.

Valve 907 is placed in a horizontal position such that a duct 908 is in fluid communication with duct 902. Vacuum is applied to a port 909 of valve 907, and the blood/reagent mixture are pulled into an incubation well 910. Once fluid contacts the hydrophobic port 906, vacuum is discontinued and port 906 is closed. Valve 901 may then be rotated 45 degrees. The blood/reagent mixture is then allowed to incubate for a predetermined amount of time.

A permanent magnet (not shown) is moved into position below well 910 for a predetermined amount of time. Consequently, targeted cells are pulled to the bottom of the well 910. Valve 907 is rotated so as to fluidly connect a wash pouch 911 containing an appropriate wash fluid to a waste well 912 having a port 913. Wash pouch 911 may be pierced and compressed. Vacuum is applied to port 913 until fluid contact therewith, and then port 913 is closed.

The permanent magnet is removed, and valve 907 is rotated to connect wash pouch 914 with a collection well 915 having a port 916. Wash pouch 914 is pierced, and vacuum is applied to port 916 of collection well 915 until fluid reaches port 916. Cells of interest are resuspended and moved into collection well 915. Cells are then ready for counting.

From the foregoing, it will be appreciated that specific embodiments of the invention have been described herein for purposes of illustration, but that various modifications may be made without deviating from the spirit and scope of the invention. Accordingly, the invention is not limited except as by the appended claims.

What is claimed is:

1. A device for facilitating volumetric blood-cell counting, comprising:
a blood reservoir configured to receive a blood sample;
a first rotatable valve including a first duct having first and second ends, the first end of the first duct configured to be selectively in fluid communication with the blood reservoir;
a first container containing a first reagent, the first end of the first duct configured to be selectively in fluid communication with the first container; and
a mixing chamber, the second end of the first duct configured to be selectively in fluid communication with the mixing chamber.

2. The device of claim 1, further comprising:
a second rotatable valve including a second duct having first and second ends, the first end of the second duct configured to be selectively in fluid communication with the second end of the first duct;
a second container containing a second reagent, the first end of the second duct configured to be selectively in fluid communication with the second container; and
a mixing chamber, the second end of the second duct configured to be selectively in fluid communication with the mixing chamber.

3. The device of claim 1, further comprising a housing, wherein the blood reservoir, first rotatable valve, first container and mixing chamber are disposed within the housing.

4. The device of claim 1, further comprising a first vacuum port in selective fluid communication with the second end of the first duct.

5. The device of claim 4, further comprising a second vacuum port in selective fluid communication with the second end of the first duct.

6. The device of claim 5, wherein the first and second vacuum ports comprise hydrophobic membranes.

7. The device of claim 1, wherein the first reagent comprises fluorescent dye.

8. The device of claim 2, wherein the second reagent comprises red blood cell lyse.
